(19) United States
(12) Patent Application Publication
(10) Pub. No.: US 2023/0357750 A1
(43) Pub. Date: Nov. 9, 2023

(54) Method for Treating a Biological Sample, and Device for Isolating Cells from a Transport Medium

(71) Applicant: Robert Bosch GmbH, Stuttgart (DE)

(72) Inventors: Franz Laermer, Weil Der Stadt (DE); Eva Weimer, Gaeufelen-Oeschelbronn (DE); Tanja Maucher, Stuttgart (DE)

(21) Appl. No.: 17/908,069

(22) PCT Filed: Mar. 8, 2021
(86) PCT No.: PCT/EP2021/055744

§ 371 Date: Aug. 30, 2022

(30) Foreign Application Priority Data
Mar. 10, 2020 (DE) 10 2020 203 035.9

(51) Int. Cl. C12N 15/10

(52) U.S. Cl. CPC C12N 15/1017

(57) ABSTRACT

The present disclosure relates to a method for treating a biological sample contained in a transport medium. At least one complexing agent is added to the transport medium, which complexing agent forms complexes with alkaline-earth metal ions. In the method, a device can be used which comprises, in succession, a porous volume filter and a membrane filter having a pore size in the range of 0.2 μm to 2.0 μm.

The present invention relates to a method for treating a biological sample in a transport medium, for example in an Amies medium. Furthermore, the present invention relates to a device for isolating cells from the transport medium.

### PRIOR ART

In diagnostics, numerous so-called transport media for biological samples, for example for bacterial samples, have become established as clinical standards. These make it possible to transport patient swabs or patient samples to a diagnostic laboratory without damaging the bacteria present in the transport medium or changing the bacterial composition in a distortive manner. Transport media provide a controlled “pleasant atmosphere” for the pathogens so that they can be introduced into a special culture medium for example at their destination and can continue to multiply. The multiplication of bacteria in a bacterial culture is an important diagnostic parameter that gives information about the identity, viability, proliferation rate and also about antibiotic sensitivities of such microbes taken from the patient. The transport medium must therefore ensure that no distortions of the later diagnosis occur at this point, for instance in that microbes that are particularly sensitive but under certain circumstances quite dangerous die prematurely and are then no longer detectable in the microbiological culture. A widely used transport medium is the so-called Amies medium.

### DISCLOSURE OF THE INVENTION

In the method for treating a biological sample present in a transport medium, at least one complexing agent that forms complexes with alkaline earth metal ions, in particular with calcium ions and with magnesium ions, is added to the transport medium. In particular, the transport medium is an Amies medium. Since Amies media exist in numerous variants, Amies media for the purposes of this method are all media that comprise calcium ions, magnesium ions, hydrogenphosphate ions and dihydrogenphosphate ions in a composition in which at least one of the solubility products of calcium hydrogenphosphate, calcium dihydrogenphosphate, magnesium hydrogenphosphate and magnesium dihydrogenphosphate is exceeded. The method is particularly advantageously suitable for treating biological samples present in an Amies medium in which all four solubility products are exceeded. The Amies medium thus comprises a suspension of alkaline earth metal hydrogenphosphates and/or alkaline earth metal dihydrogenphosphates that are partially in undissolved form as suspended matter. A problem with these compounds is the fact that they are emulsifiers that themselves are only sparingly water-soluble, if at all, but can bind water-like substances very well in their molecular structure. Such salts are therefore frequently used as emulsifiers of water in fatty phases. In particular, such calcium salts are used in foods, for example to emulsify water in sausage products. In a molecular biology procedure, this effect leads to undesirable binding of free nucleic acids to the undissolved calcium salt and magnesium salt molecule clusters if the biological sample is lyzed, with the result that the nucleic acids are subsequently no longer available for a PCR detection reaction and, since they are no longer detectable, must be considered to be lost. The effect of adding the complexing agent is that the sparingly soluble alkaline earth metal hydrogenphosphates and/or alkaline earth metal dihydrogenphosphates are converted into a dissolved form which no longer has the undesirable binding properties with respect to free nucleic acids. The complexing agent is preferably added immediately after the Amies medium has been introduced into a lab-on-chip system provided for the analysis of the biological sample. Particular preference is given to prepositioning the complexing agent in the lab-on-chip system at a suitable location and in a suitable form, for example as a solid or as a liquid reagent. A complexing agent prepositioned in solid form can for example be dissolved on contact with the Amies sample and then convert the alkaline earth metal phosphates into a soluble form by way of complex formation.

The complexing agent preferably comprises [NR¹R²R³R⁴]⁺ ions, where R¹, R², R³ and R⁴ are independently selected from hydrogen and alkyl groups. The complexing agent particularly preferably comprises ammonium ions (NH₄⁺) Ammonium ions form readily soluble diamminecalcium complexes and diamminemagnesium complexes with calcium ions and magnesium ions.

To provide ammonium ions or alkylammonium ions, the complexing agent preferably comprises at least one salt selected from the group consisting of ammonium hydrogencitrate (“binary ammonium citrate”), ammonium citrate (“ternary ammonium citrate”), ammonium thioglycolate, ammonium chloride, ammonium acetate, tetramethylammonium citrate and tetramethylammonium thioglycolate. While ammonium chloride can serve as a source of ammonium ions in a simple and cost-effective way, the other ammonium salts and tetramethylammonium salts included in the group provide additional anions that form a buffer system in the transport medium.

The transport medium, by virtue of the additions indicated, is preferably adjusted to a pH in the range of 5 to 6. The dissolution of the sparingly soluble alkaline earth metal salts is promoted even further as a result, because an acidic pH causes a shift of the hydrogenphosphates to the more soluble dihydrogenphosphates, as illustrated by the following reaction equilibrium:

H₂PO₄⁻↔HPO₄⁻+H⁺

The bacteria for example present in the biological sample still remain intact in this pH range. Although they may be greatly impaired in terms of their vitality in this pH range, what is important for subsequent molecular biology processes is merely that the cells remain intact with an undamaged cell membrane. The pH is preferably adjusted at the same time as the addition of the complexing agent or in the form of the addition of the complexing agent, since this, in the case of a suitable selection, can already have the aforementioned buffer effect on its own. In this embodiment of the method, the complexing agent simultaneously functions as a buffer. This is the case for example for ammonium hydrogencitrate, the so-called “binary ammonium citrate” (NH₄)₂H citrate, that on its own sets a pH of 5 to 6. If “ternary ammonium citrate” is used instead, this being (NH₄)₃ citrate, then an approximately neutral pH of approx. 7 is brought about. A further addition of citric acid then makes it possible, in the combination as a buffer system, to establish the desired pH range between 5 and 6.

It is preferable for a citric acid/citrate buffer and/or an acetic acid/acetate buffer and/or a thioglycolic acid/thioglycolate buffer to be added to the transport medium. These buffers are particularly suitable for setting a pH in the range of 5 to 6. If the intention is to use a citric acid/citrate buffer, then the complexing agent is preferably ammonium hydrogencitrate, ammonium citrate or tetramethylammonium citrate, and citric acid is additionally added thereto. If the buffer is intended to be an acetic acid/acetate buffer, then the complexing agent is preferably ammonium acetate, and acetic acid is added thereto. If the intention is to use a thioglycolic acid/thioglycolate buffer, then the complexing agent is preferably ammonium thioglycolate or tetramethylammonium thioglycolate, and thioglycolic acid, also known as mercaptoacetic acid, is added thereto. It is also possible in principle for the complexing agent to merely comprise one of the salts suitable for buffer formation, i.e. a hydrogencitrate, a citrate, an acetate or a thioglycolate, and for the corresponding acid to then be released therefrom by addition of another organic acid. Mandelic acid is suitable in particular for this purpose. The addition of a further organic acid, such as mandelic acid, to an existing system consisting of salt and corresponding acid may also be envisaged.

It is possible only with great difficulty to establish sufficiently chaotropic conditions in transport media as would be required for binding nucleic acids to a filter frit, without diluting the sample to a very great extent. This leads either to a restriction of the amount of sample material to be processed, for example with respect to limited capacity of a lab-on-chip cartridge and amounts of reagents used and thereby a restricted amount of analytes to be detected, or to a huge loss of DNA by way of insufficiently chaotropic conditions with the consequence of poor nucleic acid binding on the filter frit. Both have the consequence of poor detection sensitivity. In order to tackle this problem, it is preferable in the method, after adding the complexing agent, for the transport medium to first be filtered through a porous volume filter. It is then filtered through a membrane filter with a pore size in the range of 0.2 μm to 2.0 μm. The two filters are preferably combined by stacking one on top of the other in the form of a single filter stack. The pore size of the membrane filter is particularly preferably in the range of 0.4 μm to 1.2 μm and very particularly preferably in the range of 0.8 μm to 1.0 μm. The statement of a numerical value of a pore size always relates here to the membrane filter and not to the upstream porous volume filter. This pore size makes it possible for the membrane filter, in conjunction with the upstream porous volume filter, to readily hold back cells, such as bacterial cells, without becoming clogged. Other substances, such as dissolved mucus or blood components or other impurities from the biological sample, pass through the filter arrangement or are already held back by the volume filter without clogging the membrane filter. In particular, the porous volume filter upstream of the membrane filter may have a constant porosity or a sequence of porosity values that decrease toward the membrane filter. This makes it possible to hold back suspended matter present in the transport medium, such as activated carbon or sample impurities, so that they cannot clog the membrane filter and only the cells from the biological sample collect on the membrane filter.

For the further treatment of the biological sample, it is preferable for cells that remain on the membrane filter to be freed of alkaline earth metal hydrogenphosphates, alkaline earth metal dihydrogenphosphates and impurities from the transport medium. Since these have been converted into a soluble form by the addition of the complexing agent, this can be achieved in a simple manner by washing by means of a wash buffer. For this purpose, use can in particular be made of a polysorbate/water mixture. This also washes off other residues from the transport medium and remaining small impurities from the biological sample, such as mucus.

In order to release the nucleic acids from the remaining cells, it is preferable for said cells to be lyzed on the membrane filter. Suitable lysis methods are in particular heating to a temperature of preferably more than 60° C. and/or adding a lysis medium such as in particular octoxynol-9 and/or mechanical stress, in particular by ultrasound exposure.

In order to be able to carry out a PCR detection reaction with the released nucleic acids, said nucleic acids are preferably eluted from the membrane filter. This can be performed both during the lysis and subsequent to the lysis. If the elution is to be performed at the same time as the lysis, then the lysis medium simultaneously functions as eluent. In the case of a subsequent elution, the nucleic acids are flushed out of the membrane filter by means of a suitable eluent after the lysis. Once the eluate has been mixed with a PCR master mix, for example in the form of a lyophilized PCR bead prepositioned in a lab-on-chip, a PCR amplification and detection reaction can then take place.

Use can be made in this method of a device for isolating cells from a transport medium that has, in succession, a porous volume filter and a membrane filter with a pore size in the range of 0.2 μm to 2.0 μm. The pore size of the membrane filter is preferably in the range of 0.4 to 1.2 μm and particularly preferably in the range of 0.8 to 1.0 μm.

### BRIEF DESCRIPTION OF THE DRAWINGS

FIG. 1 shows a flow diagram of an exemplary embodiment of the method according to the invention.

FIG. 2 shows a schematic representation of an exemplary embodiment of the device according to the invention.

### EXEMPLARY EMBODIMENTS OF THE INVENTION

In one exemplary embodiment of the invention, a biological sample, which is a bacterial sample in the form of a patient swab, is introduced into an Amies medium as transport medium. In the present exemplary embodiment, the Amies medium comprises
0.2 g/l potassium chloride
0.2 g/l potassium dihydrogenphosphate
0.1 g/l calcium chloride
3.0 g/l sodium chloride
1.15 g/l sodium hydrogenphosphate
1.0 g/l sodium thioglycolate
0.1 g/l magnesium chloride
6.5 g/l agar

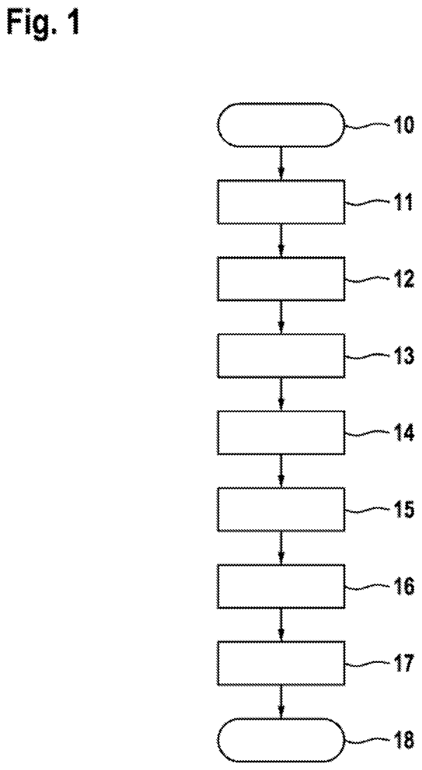

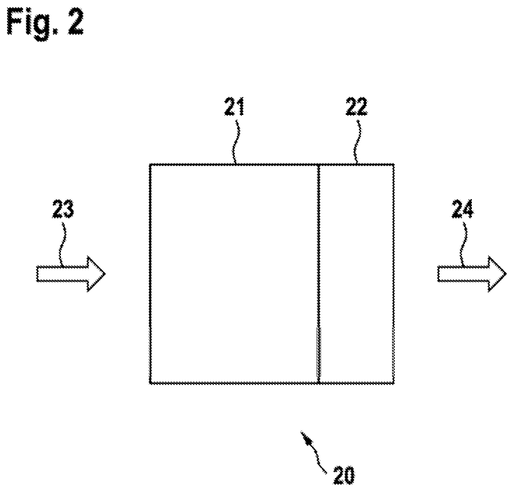

It has a pH of around 7.3. Once the Amies medium with the biological sample has been transported to a diagnostic laboratory, it is treated there by means of an exemplary embodiment of the method according to the invention. As shown in FIG. 1, after the method has started 10, the Amies medium with the biological sample is inserted 11 into a lab-on-chip system. In this, a complex-forming buffer mixture is added 12 to the Amies medium. In the present exemplary embodiment, the complex-forming buffer mixture comprises ammonium hydrogencitrate and citric acid in a mixing ratio which buffers the Amies medium to a pH in the range of 5 to 6. Calcium dihydrogenphosphate, calcium hydrogenphosphate, magnesium dihydrogenphosphate and magnesium hydrogenphosphate, formed from the chlorides of the two alkaline earth metal elements along with the potassium dihydrogenphosphate and the sodium hydrogenphosphate, are suspended in the Amies medium and go back into solution with formation of diamminecalcium complexes and diamminemagnesium complexes. The Amies medium thus treated is then introduced into a device 20 for isolating cells that is arranged in the lab-on-chip and is shown in FIG. 2.

The device 20 has a porous volume filter 21. Arranged immediately downstream of this is a porous membrane filter 22 that has a pore size of 0.9 μm in the present exemplary embodiment. The Amies medium is introduced into the device 20 at an introduction position 23 on the volume filter 21. The Amies medium is first filtered 13 through the volume filter 21 as a result. This holds back agar and relatively large particles or sample impurities in the volume filter 21, while bacterial cells easily pass through this volume filter and thus reach the membrane filter 22. The Amies medium is then further filtered 14 through the membrane filter 22. Here, the bacterial cells from the biological sample remain on the membrane filter 22, while all soluble components of the Amies medium leave the device 20 through the membrane filter 22 and a subsequent exit position 24 as an aqueous solution.

The cells are then washed 15 on the membrane filter 22 by introducing a Tween®/water mixture (polysorbate/water mixture) into the device 20 through the inlet point 23. This washes remaining soluble constituents of the Amies medium and soluble constituents of the biological sample still adhering to the surface of the cells out of the device 20 through the membrane filter 22 and through the outlet point 24. Afterward, the cells are lyzed 16 by introducing a 0.1 wt % solution of Triton® X-100 (octoxynol-9) into the device 20 through the introduction position 23. This functions as a lysis medium and simultaneously elutes the free nucleic acids from the bacteria, obtained by way of the lysis, out of the device through the outlet opening 24. The eluate is mixed with a PCR master mix in the form of a lyophilized PCR bead and a PCR detection reaction is carried out 17. The method is then ended 18.

1. A method for treating a biological sample present in a transport medium, comprising:
adding at least one complexing agent that forms complexes with alkaline earth metal ions to the transport medium.

2. The method as claimed in claim 1, wherein the transport medium is an Amies medium.

3. The method as claimed in claim 1, wherein the alkaline earth metal ions are at least one of calcium ions and/or magnesium ions.

4. The method as claimed in claim 1, wherein the at least one complexing agent comprises [NR¹R²R³R⁴]⁺ ions, where R¹, R², R³ and R⁴ are independently selected from H and alkyl groups.

5. The method as claimed in claim 4, wherein the at least one complexing agent comprises at least one salt selected from a group consisting of ammonium hydrogencitrate, ammonium citrate, ammonium thioglycolate, ammonium chloride, ammonium acetate, tetramethylammonium citrate, and tetramethylammonium thioglycolate.

6. The method as claimed in claim 1, further comprising:
adjusting the transport medium to a pH in the range of 5 to 6.

7. The method as claimed in claim 6, further comprising:
adding at least one of a citric acid/citrate buffer, an acetic acid/acetate buffer, and/or a thioglycolic acid/thioglycolate buffer to the transport medium.

8. The method as claimed in claim 1, further comprising:
filtering, after adding the complexing agent, the transport medium through a porous volume filter; and
filtering the filtered transport medium through a membrane filter with a pore size in the range of 0.2 μm to 2.0 μm.

9. The method as claimed in claim 8, characterized in that further comprising:
freeing cells that remain on the membrane filter of alkaline earth metal hydrogenphosphates, alkaline earth metal dihydrogenphosphates and impurities from the transport medium.

10. The method as claimed in claim 8, further comprising:
lyzing cells that remain on the membrane filter.

11. The method as claimed in claim 10, further comprising:
eluting nucleic acids released during the lysis from the membrane filter.

12. A device for isolating cells from a transport medium, comprising:
a porous volume filter; and
a membrane filter with a pore size in the range of 0.2 μm to 2.0 μm located downstream of the porous filter.
